(19) United States
(12) Patent Application Publication
(10) Pub. No.: US 2024/0300869 A1
(43) Pub. Date: Sep. 12, 2024

(54) MICROBIAL AGENT FOR PROMOTING ROOT NODULE NUMBER INCREASE AND ROOT NODULE NITROGENASE ACTIVITY INCREASE IN LEGUMINOUS CROPS AND USE THEREOF

(71) Applicant: OIL CROPS RESEARCH INSTITUTE, CHINESE ACADEMY OF AGRICULTURAL SCIENCES, Hubei (CN)

(72) Inventors: Peiwu LI, Hubei (CN); Xiaofeng YUE, Hubei (CN); Qi ZHANG, Hubei (CN); Xiaoqian TANG, Hubei (CN); Yang ZHOU, Hubei (CN); Yizhen BAI, Hubei (CN); Jun JIANG, Hubei (CN)

(73) Assignee: OIL CROPS RESEARCH INSTITUTE, CHINESE ACADEMY OF AGRICULTURAL SCIENCES, Hubei (CN)

(21) Appl. No.: 18/664,304

(22) Filed: May 15, 2024

Related U.S. Application Data
(63) Continuation of application No. PCT/CN2022/131941, filed on Nov. 15, 2022.

(30) Foreign Application Priority Data
Nov. 15, 2021 (CN) 202111344653.8

(51) Int. Cl. C05F 11/08; C05G 5/12

(52) U.S. Cl. CPC C05F 11/08; C05G 5/12

(57) ABSTRACT

The present disclosure belongs to the field of microbial technology, and in particular relates to a microbial agent for promoting root nodule number increase and root nodule nitrogenase activity increase in leguminous crops and use thereof. The microbial agent is compounded from 4 microbes including Bacillus amyloliquefaciens, Brevibacillus laterosporu, Bacillus mucilaginosus Krassilnikov, and Enterobacter ludwigii by separate fermentative cultivation, concentration and mixing. The microbial agent can effectively promote root nodule number increase and root nodule nitrogenase activity increase, promote crop growth, and improve crop yield and product quality.

### CROSS-REFERENCE TO RELATED APPLICATION

The present application is a continuation of PCT application No. PCT/CN2022/131941, filed on Nov. 15, 2022, which claims the priority benefits of China Application No. 202111344653.8, filed on Nov. 15, 2021. The entirety of each of the above mentioned patent applications is incorporated by reference herein and made a part of this specification.

### BACKGROUND

### Technical Field

The present disclosure belongs to the field of microbial technology, and in particular relates to a microbial agent for promoting root nodule number increase and root nodule nitrogenase activity increase in leguminous crops and use thereof.

### Description of Related Art

Roots of Leguminous crops such as peanuts can coexist with rhizobia in soil for nodulation and nitrogen fixation. When the roots of the peanuts germinate and grow, the rhizobia invade through root cracks, stimulating the development of root cortical cells, and gradually forming macroscopic root nodules on taproots and lateral roots. The rhizobia colonize and reproduce in large quantities in the root nodules, forms many spherical bacteroids at a certain period, and performs biological nitrogen fixation by the action of nitrogenase, converting nitrogen in the air into a nitrogen fertilizer, and providing nitrogen nutrition for the leguminous crops, which not only improves soil fertility, promotes high crop yield, but also reduces chemical fertilizer input, and reduces environmental pollution. Therefore, the biological nitrogen fixation plays an important role in improving soil nutrition and solving the problem of sustainable development of agriculture and energy.

However, excessive dependency on chemically synthesized fertilizers, and long-term excessive or unreasonable use in agricultural practical production cause soil hardening, trace element deficiency, and soil nutrient imbalance, which inhibits growth, propagation and nodulation of rhizobia and nitrogen-fixing bacteria in soil, resulting in a low number of root nodules and low biological nitrogen fixation efficiency in the leguminous crops such as peanuts. In addition, the leguminous plants have high specificity in symbiosis with the rhizobia, the number of effective rhizobia in newly planted soil is often small, and high-efficiency and excellent strains need to be artificially inoculated to fully exert the nitrogen fixation effect. A microbial agent is an environment-friendly fertilizer, and can increase soil fertility, facilitate propagation of beneficial microorganisms in soil, optimize the microecological environment, improve the biological nitrogen fixation capacity of leguminous crops, and promote yield increase. Therefore, the screening of high-efficiency nitrogen-fixing microorganisms and the application of nodule nitrogen-fixing microbial fertilizers have always been hot spots in the field of biological nitrogen fixation research, and in recent years, microbial fertilizers have developed rapidly and varieties have continued to increase. However, due to the types of strains, nitrogen fixation capacity, production technology, living bacteria count, and the like, the effect of the microbial fertilizer is not obvious. At present, there are no products that can be applied to production on a large scale and have significant nodulation and nitrogen fixation effects, especially can induce “supernodulation” in leguminous crops.

To sum up, an object of the present disclosure is to develop and apply a microbial agent in view of the deficiencies in the prior art, aiming at efficiently promoting an increase of the number of root nodules in leguminous crops and improving the biological nitrogen fixation effect. The microbial agent of the present disclosure is mainly developed from 4 exogenous and endophytic strains of Bacillus amyloliquefaciens, Brevibacillus laterosporu, Bacillus mucilaginosus and Enterobacter ludwigii (CN105586300B) isolated from peanut pods and rhizospheres in main peanut production areas of China, and has the effects of promoting a significant increase of the number, weight, and density of root nodules in leguminous crops such as peanuts, enhancing the nitrogenase activity in root nodules, increasing the chlorophyll content in leaves, increasing the nitrogen content in plants and total biomass.

### SUMMARY

In view of the deficiencies in the prior art, the technical problem to be solved by the present disclosure is to provide a microbial agent for promoting root nodule number increase and root nodule nitrogenase activity increase in leguminous crops and use thereof in order to increase the nodulation and nitrogen fixation effect of leguminous crops such as peanuts. The microbial agent can effectively promote root nodule number with the nitrogenase activity increase and root nodule nitrogenase activity increase, promote crop growth, and improve crop yield and product quality.

To solve the above technical problems, the technical solutions adopted by the present disclosure are as follows:
provided is a microbial agent for promoting root nodule number increase and root nodule nitrogenase activity increase in leguminous crops, compounded from 4 microbes including Bacillus amyloliquefaciens, Brevibacillus laterosporu, Bacillus mucilaginosus Krassilnikov, and Enterobacter ludwigii by separate fermentative cultivation, concentration and mixing.

Further, the Bacillus amyloliquefaciens is a Bacillus amyloliquefaciens BA-HZ54 strain with an accession number of CCTCC No. M 20211295, which is preserved on Oct. 20, 2021 at the China Center for Type Culture Collection (abbreviated as CCTCC) of Wuhan University, Wuhan, China.

The Brevibacillus laterosporu is a Brevibacillus laterosporu BL-TS08 strain with an accession number of CCTCC NO: M 20211296, which is preserved on Oct. 20, 2021 at the China Center for Type Culture Collection (abbreviated as CCTCC) of Wuhan University, Wuhan, China.

The Bacillus mucilaginosus Krassilnikov is a Bacillus mucilaginosus Krassilnikov BM-TS05 strain with an accession number of CCTCC NO: M 20211297, which is preserved on Oct. 20, 2021 at the China Center for Type Culture Collection (abbreviated as CCTCC) of Wuhan University, Wuhan, China.

Further, the Enterobacter ludwigii is an Enterobacter ludwigii BG10-1 strain with an accession number of CCTCC NO: M 2016014, which is preserved on Jan. 7, 2016 at the China Center for Type Culture Collection of Wuhan University (CN201610155898.9).

Preferably, the microbial agent has a living bacteria count of the Bacillus amyloliquefaciens being 2×10⁹ cfu/g or more, a living bacteria count of the Brevibacillus laterosporu being 2×10⁹ cfu/g or more, a living bacteria count of the Bacillus mucilaginosus Krassilnikov being 1×10¹⁰ cfu/g or more, and a living bacteria count of the Enterobacter ludwigii being 1×10¹⁰ cfu/g or more.

The above four microbial strains are isolated from peanut pods and rhizospheres in main peanut production areas of China.

Further, the microbial agent according to the present disclosure is a highly concentrated live bacterial granule or powder or aqueous agent, preferably the granule.

According to the above solution, a carrier of the granule includes humic acid, tapioca flour and a bentonite binder. A weight ratio of the humic acid to the tapioca flour to the bentonite binder is about 8.5:10:0.5, and raw materials are uniformly mixed and granulated and oven-dried to obtain a granular carrier for standby application.

According to the above solution, the microbial agent of the present disclosure is produced by compounding concentrated bacterial solutions of strains with the particle carrier. Specifically, concentrated thalli obtained by centrifugation of fermentation broths of the strains are dissolved in an appropriate amount of water to be uniformly sprayed and adsorbed onto the particle carrier.

Provided is use of the microbial agent in promoting root nodule number with the nitrogenase activity increase and root nodule nitrogenase activity increase in leguminous crops.

According to the above solution, a specific application method includes applying the microbial agent at a usage amount of 2-4 kg/mu at a sowing stage or a growth period of the leguminous crops. In particular, the microbial agent can be applied alone or mixed with a base fertilizer to be subjected to artificial broadcast application/hole application, or mechanically spread onto soil.

According to the above solution, the leguminous crops include, but are not limited to, peanuts, soybeans, alfalfa, Chinese milk vetch and the like.

According to the above solution, the use further includes inducing supernodulation of the leguminous crops.

The beneficial effects of the present disclosure are as follows:
1. The microbial agent of the present disclosure can effectively promote the increase of the root nodules and the enhancement of the nitrogenase activity in the root nodules in the leguminous crops such as peanuts, soybeans, alfalfa, and Chinese milk vetch, induce supernodulation in roots, significantly improve the nitrogenase activity and the biological nitrogen fixation effect of roots, and increase the nitrogen nutrient supply capacity of leguminous crops, and has the beneficial effects of promoting crop growth, increasing the chlorophyll content, nitrogen content and dry matter accumulation in plants, and finally improving crop yield and quality, having significant economic benefits.
2. The microbial agent of the present disclosure can be applied alone or applied with a fertilizer during the sowing stage, is simple and convenient to use, is easy to promote, and has a small usage amount per mu (only 2-4 kg/mu), significantly saving the cost and increasing the efficiency.
3. The microbial agent of the present disclosure not only avoids environmental pollution and energy waste caused by excessive chemical fertilizer input, but also facilitates improving soil fertility, and improving the farmland ecological environment, has significant ecological benefits, and is of great significance for reducing fertilizer application, peak carbon dioxide emissions and carbon neutrality.

### BRIEF DESCRIPTION OF THE DRAWINGS

FIG. 1: Microbial agent for promoting an increase of the number of root nodules in leguminous crops.

FIG. 2: Comparison of root nodules of peanuts in a control area and a microbial agent treatment area.

FIG. 3: Comparison of growth vigour of control peanuts and peanuts treated with a microbial agent.

FIG. 4: Comparison of the nitrogenase activity in root nodules of control peanuts and

treated peanuts.

FIG. 5: Comparison of the fresh weight of control peanut plants and treated peanut plants per square meter.

FIG. 6: Comparison of the dry weight of a single control peanut plant and a single treated peanut plant.

FIG. 7: Comparison of the number and weight of root nodules in the single control peanut plant and the single treated peanut plant.

### DESCRIPTION OF THE EMBODIMENTS

### Example 1: Preparation Method for a Microbial Agent for Promoting an Increase of the Number of Root Nodules in Leguminous Crops

### 1. Identification of Strains

Microbial strains involved in the present disclosure were obtained from peanut pods and rhizospheres in Tangshan, Hebei and Hezhou, Guangxi by conventional bacterial isolation and purification, and molecular identification of a 16S rDNA sequence. Wherein a Bacillus amyloliquefaciens BA-HZ54 strain with an accession number of CCTCC NO: M 20211295, a Brevibacillus laterosporu BL-TS08 strain with an accession number of CCTCC NO: M 20211296 and a Bacillus mucilaginosus Krassilnikov BM-TS05 strain with an accession number of CCTCC NO: M 20211297 were preserved on Oct. 20, 2021 at the China Center for Type Culture Collection (abbreviated as CCTCC) of Wuhan University. Enterobacter ludwigii was preserved on Jan. 7, 2016 at the China Center for Type Culture Collection of Wuhan University (Chinese Patent application Ser. No. 201610155898.9), with an accession number of CCTCC NO: M 2016014.

### 2. Preparation of the Microbial Agent

1) Microbial fermentation production. The activated Bacillus amyloliquefaciens, Brevibacillus laterosporu, Bacillus mucilaginosus Krassilnikov, and Enterobacter ludwigii were separately inoculated into a liquid medium (containing 3.5-4.0% corn flour, 1.5-2.0% peptone, 0.4-0.5% K₂HPO₄+KH₂PO₄ (1:1), and 1 L of water, having a pH of 7.0-7.2, and being sterilized at 121° C. for 20 min) under aseptic conditions, and shaking culture was performed in a triangular flask at 180 r/min at 37° C. for 24 h. The cultured strains were then separately inoculated into a 300 L fermentation tank at an inoculation amount of 1% for fermentation production. The fermentative cultivation was carried out at a temperature of 30-37° C., a pH of 7.0-7.2, and a stirring speed of 180-220 rpm, and the fermentation was stopped after the amount of thalli reached 1×10¹⁰ cfu/ml.

2) Preparation of a particle carrier of the microbial agent. The particle carrier consists of humic acid, tapioca flour, and a bentonite binder. The raw materials were present in the particle carrier in a ratio of 8.5:10:0.5. The raw materials were uniformly mixed and granulated by a granulator and oven-dried for standby application.

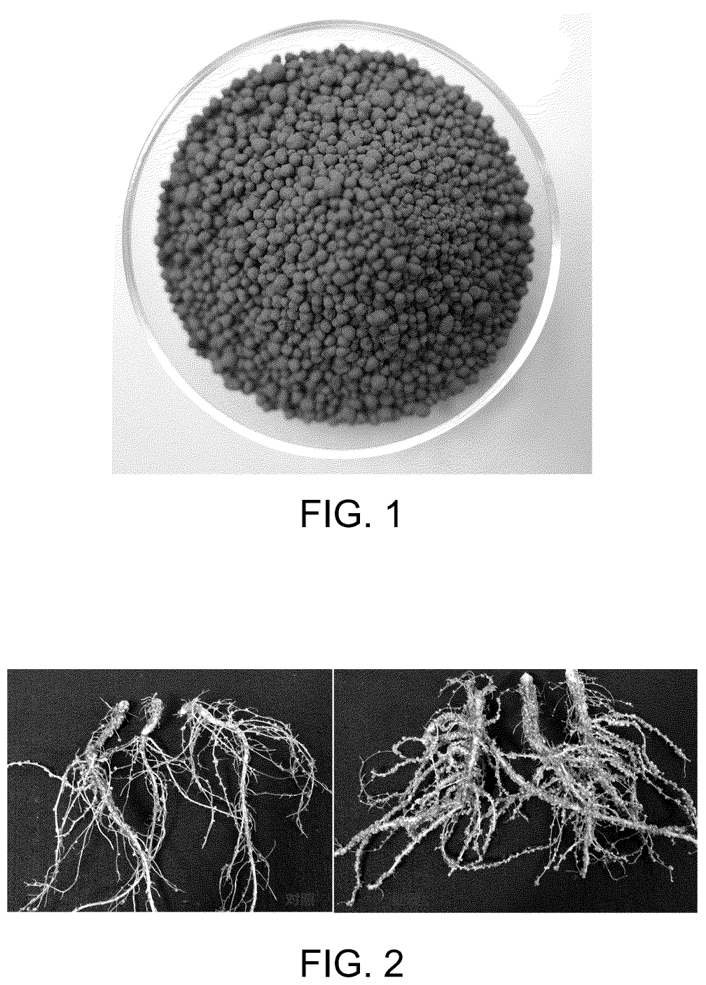

3) Preparation of a microbial agent product. According to a ratio of fermentation broths of 200 mL of Bacillus amyloliquefaciens+200 mL of Brevibacillus laterosporu+1 L of Bacillus mucilaginosus Krassilnikov+1 L of Enterobacter ludwigii for producing 1 kg of a microbial fertilizer, thalli obtained by centrifugation of four strain fermentation broths with determined volumes were dissolved in an appropriate amount of water to prepare a mixed bacterial suspension, then the carrier was sprayed in a mixer (a mass ratio of the bacterial suspension to the carrier was 1:10), and drying was performed at a low temperature (≤60° C.) to prepare the microbial agent (see FIG. 1), and the microbial agent was packaged to obtain a finished microbial agent product. The microbial agent developed has a living bacteria count of the Bacillus amyloliquefaciens being 2×10⁹ cfu/g or more, a living bacteria count of the Brevibacillus laterosporu being 2×10⁹ cfu/g or more, a living bacteria count of the Bacillus mucilaginosus Krassilnikov being 1×10¹⁰ cfu/g or more, and a living bacteria count of the Enterobacter ludwigii being 1×10¹⁰ cfu/g or more.

### Example 2: Effect of Application of the Microbial Agent in Promoting an Increase of the Number of Root Nodules in Leguminous Crops Such as Peanuts

### 1. Field Trial Setup

Field demonstration application of the microbial agent product was carried out in main production areas of leguminous crops such as peanuts in Zhengyang, Henan, Tangshan, Hebei, Fuxin, Liaoning, Xiangyang, Hubei, and the like of China, with a test area of 50 mu at each test point, a control group and a treatment group were set, and an isolation zone was set between the treatment group and the control group.

Control group: an area was 25 mu, a variety was a local main variety, and a local conventional sowing method, and conventional field management measures such as weeding, pest control, and vigorous growing control were used.

Treatment group: an area, a variety, a sowing method, and a field management technique were all the same as those in the control group, and on this basis, the microbial agent product was uniformly mixed with a base fertilizer at a usage amount of 2 kg/mu to be evenly spread onto soil with the fertilizer either mechanically or manually during a peanut sowing stage.

### 2. Nodulation Investigation and Determination

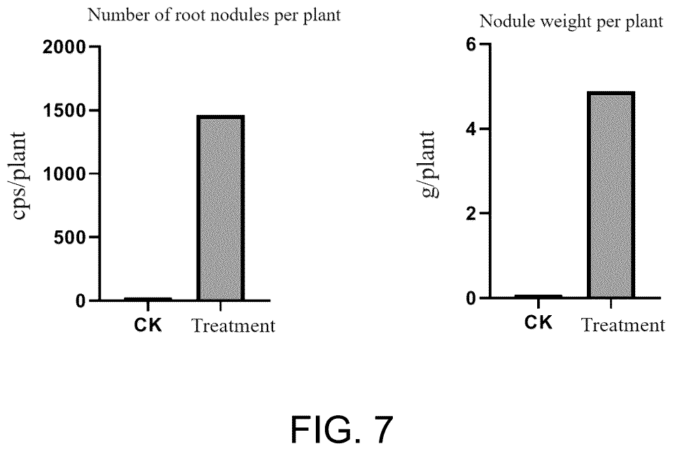

Compared with the control group, the treatment group has the following advantages: (1) peanuts have developed root systems, stable growth vigour, and do not age prematurely; (2) the number of root nodules has significantly increased by 50 times or more; and root systems and nodulation statuses were investigated before peanut harvesting, and the number, weight and density of root nodules were determined (at least 5 plant samples for each of the control group and the treatment group at different test points). The results are shown in FIG. 2 and FIG. 7: \textcircled{1} compared with the control group, peanuts had more developed root systems, and more stable growth vigour; \textcircled{2} microbial agent treatment significantly promoted an increase in the number of root nodules at each test point, there were rare peanut root nodules in the control group, and there were more and dense peanut root nodules in the treatment group; and \textcircled{3} the number and weight of root nodules per plant significantly increased by 50 times or more after microbial agent treatment. The above results show that the number, weight and density of root nodules are significantly increased in the microbial agent treatment group compared with the control group, and the microbial agent of the present disclosure can effectively promote the nodulation and nitrogen fixation in root systems of leguminous crops such as peanuts.

### Example 3: Effect of Application of the Microbial Agent in Increasing the Nitrogenase Activity in Root Nodules of Leguminous Crops Such as Peanuts

The field trial setup was the same as that in Example 2.

The nitrogenase activity reflects the strength of the nitrogen-fixing ability of root nodules, and in order to further clarify whether root nodules of peanuts treated with the microbial agent have the nitrogenase activity, the nitrogenase activity in the root nodules in the control group and the treatment group was detected by an acetylene reduction method recognized by those skilled in the art, and specific steps were as follows.

1) Parameter setting of gas chromatography: peak values of ethylene and acetylene were detected by a hydrogen flame ionization detection method (FID) in the present disclosure. Operation steps were detailed in the instrument instructions. Operating parameters of a domestic GC-4000 Series gas chromatograph were as follows: vaporization chamber temperature: 120° C.; chromatography chamber temperature: 60° C.; hydrogen flame: 130° C.; column pressure: about 0.1 Mpa; air pressure: about 0.2 Mpa, and hydrogen pressure: about 0.05 Mpa; carrier gas (N₂) pressure: about 0.3 Mpa; attenuation: 1× or 2×, and column: GDX-502.

During detection, 50-100 μL of a gas sample was sucked with a micro sampling syringe, and the retention time of ethylene in a chromatographic column was 1.5-2.5 min.

2) Detection of the nitrogenase activity in root nodules: an appropriate amount of root nodules were placed in a vial, a mouth of the vial was sealed with a rubber stopper, and 5 or more replicates were set for each experimental treatment. A 1/10 volume of air was extracted from the vial with a syringe, causing a negative pressure inside the vial, and a 1/10 volume of acetylene gas was injected as a substrate for nitrogenase. The vial was placed at 28° C. for a reaction for 2 h, and then the vial was taken out for detection.

3) Ethylene standard curve making: 6 glass vials of the same volume were taken, and a certain volume of ethylene gas was separately added into the glass vials by using a micro sampling syringe to detect the peak value of ethylene. The selection of an ethylene volume gradient depends on the ethylene reducing ability of root nodule samples. The principle is that the range of peak area values of the standard curve covers peak area values of all the samples to be measured when the samples and the standard curve are measured under the same conditions of the chromatograph.

Ethylene volume=K×peak area

K is a response coefficient and can be calculated by linear regression of ethylene volume values and corresponding peak area values in the standard curve.

4) Enzyme activity calculation:

Nitrogenase activity is the number of moles of acetylene reduced per unit time by root nodules per unit weight:

Nitrogenase activity=the number of moles of acetylene/fresh nodule weight×reaction time

According to the relationship between the number of moles of gas and the volume, temperature, and pressure, the number of moles can be obtained from the volume of ethylene:

$${C}_{2} {H}_{4} μ mol = {C}_{2} {H}_{4} volume ( μL ) \times \frac{1}{22.4} \times \frac{273}{( 273 + t ° C )} \times P / 760$$

in the formula: t° C.: Celsius air temperature; P: gas pressure, typically taken to be 760 mm Hg;

22.4: the volume of 1 mol gas in a standard state is 22.4 L; 273 is the absolute temperature

The results showed that microbial agent treatment efficiently promoted nodulation in peanut roots, and these root nodules all had nitrogenase activity, and the nitrogenase activity per gram of root nodules and the nitrogenase activity in roots per plant significantly increased after microbial agent treatment (FIG. 4). The range of the nitrogenase activity per gram of root nodules of control peanuts was 0.027-0.68 μmol/g·h, with an average value of 0.36 μmol/g·h, and the range of the nitrogenase activity per gram of root nodules of peanuts in the treatment group was 1.19-2.98 μmol/g·h, with an average value of 2.07 μmol/g·h. The nitrogenase activity per gram of the root nodules of the peanuts in the treatment group was improved by 5 times or more compared with the control group. The range of the nitrogenase activity in roots per control peanut plant was 0.020-0.33 μmol/plant·h, with an average value of 0.18 μmol/plant·h, and the range of the nitrogenase activity in roots per peanut plant in the treatment group was 4.51-10.60 μmol/plant·h, with an average value of 7.10 μmol/plant·h. The nitrogenase activity in roots per peanut plant in the treatment group was increased by 40 times.

The results show that the microbial agent of the present disclosure can significantly enhance the nitrogenase activity in the root nodules of leguminous crops such as peanuts, and improve the biological nitrogen fixation capacity and nitrogen fixation effect of the root nodules, and has important practical significance for nitrogen nutrient supply of leguminous crops such as peanuts, reduction of fertilizer application in farmlands, peak carbon dioxide emissions and carbon neutrality, and the like.

### Example 4: Application of the Microbial Agent to Increase the Chlorophyll Content in Leguminous Crops Such as Peanuts

The field trial setup was the same as that in Example 2.

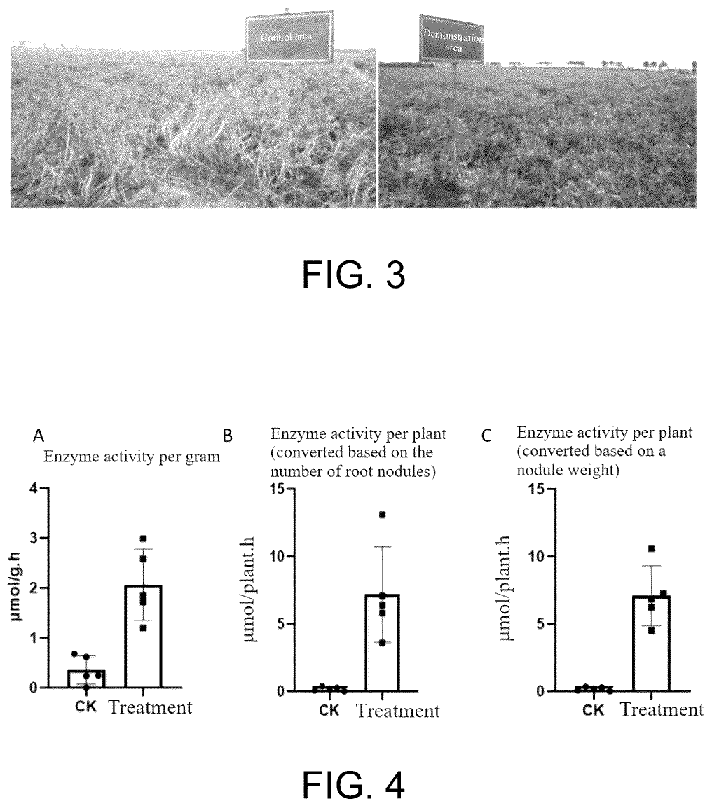

During the growth period of peanuts, the growth vigour and leaf conditions of the plants were investigated, and it was found that compared with the control area, the growth vigour of the peanuts was stronger, and the leaves were greener in the microbial agent treatment area (FIG. 3). Peanut leaves were collected during the peanut growth period, and the chlorophyll content in leaves from test points such as Henan, Jilin, Hubei, Hunan, and Sichuan was determined by spectrophotometry (3 samples were randomly collected from each test point, and each sample was formed by mixing at least 5 single plant leaves). The steps of sample processing and detection methods specifically refer to “Rapeseed-Determination of chlorophyll content-Spectrometric method” in a national standard GB/T 22182-2008.

The comparison results of the chlorophyll content after microbial agent treatment are shown in Table 1. The chlorophyll content of the peanut leaves in the control group was 447.49-498.16 mg/kg, with an average value of 471.57 mg/kg, and the chlorophyll content of the peanut leaves after treatment with the microbial agent of the present disclosure was 492.70-531.39 mg/kg, with an average value of 511.54 mg/kg. The microbial agent treatment significantly increased the chlorophyll content in peanut leaves, which was improved by 5.45%-13.39% compared with the control group, with an average increase of 8.48%.

The results show that the microbial agent of the present disclosure has the effects of improving crop nitrogen nutrition, and increasing the chlorophyll content and nitrogen content in leaves, thus increasing the protein content, and improving crop nutritional quality.

TABLE 1
Comparison of the chlorophyll content
after microbial agent treatment

| | | |
|---|---|---|
| Test point | Average (mg/kg) | Increase |

| and code | Control | Treatment | rate (%) |
|---|---|---|---|
| Jilin JLFY | 498.16 | 525.33 | 5.45 |
| Henan HNZY | 468.65 | 531.39 | 13.39 |
| Hubei HBXY | 447.49 | 502.07 | 12.20 |
| Hunan HNAH | 477.70 | 506.23 | 5.97 |
| Sichuan SCCD | 465.87 | 492.70 | 5.76 |
| Average | 471.57 | 511.54 | 8.48 |

### Example 5: Application of the Microbial Agent to Increase Biomass and Dry Matter Accumulation in Leguminous Crops Such as Peanuts

The field trial setup was the same as that in Example 2.

The aboveground fresh weight, peanut pod fresh weight and dry weight of peanut plants per square meter (3 points were randomly selected for control and treatment per test point, with 2 square meters for sampling and determination at each point) and single plants (10 or more plants were randomly selected for control and treatment per test point) during the harvest period were investigated and weighed.

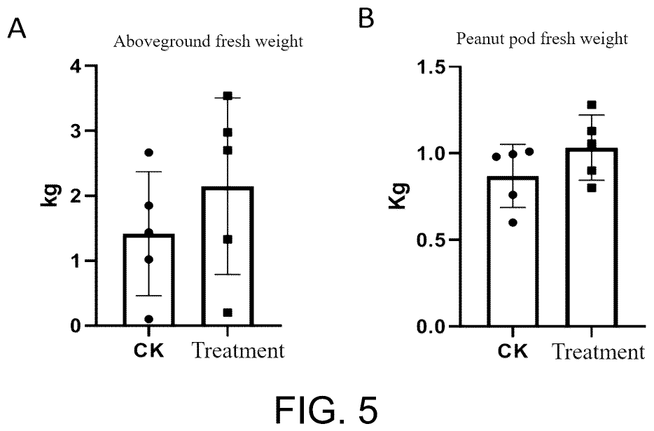

The determination results of the aboveground fresh weight and underground fresh weight of peanuts per square meter at demonstration application points in main peanut production areas such as Liaoning, Jilin, Henan, Jiangsu, and Hubei are shown in FIG. 5: at each test point, the range of the fresh weight content of peanut aboveground plants per square meter in the control area was 0.1-2.67 Kg, with an average value of 1.42 Kg, and the range of the fresh weight content of peanut aboveground plants per square meter in the treatment area was 0.2-3.54 Kg, with an average value of 2.15 Kg, which was 1.5 times that of the control area; and at each test point, the range of the fresh weight content of peanut pods per square meter in the control area was 0.6-1.01 Kg, with an average value of 0.86 Kg/m², and the range of the fresh weight content of peanut pods per square meter in the treatment area was 0.8-1.28 Kg, with an average value of 1.03 Kg, which was 1.2 times that of the control area.

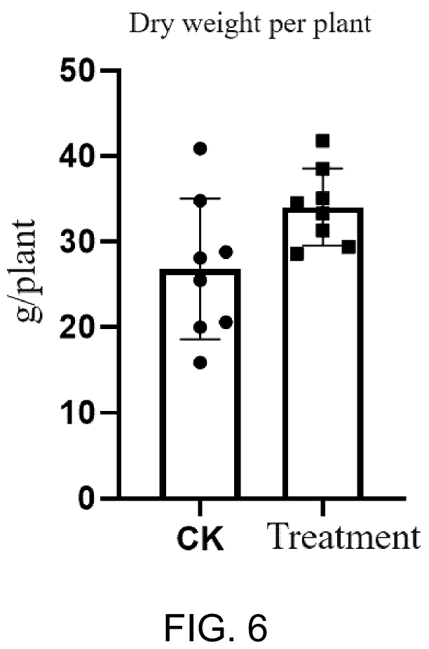

The aboveground dry weight per peanut plant was determined, and the results are shown in FIG. 6: the aboveground dry weight per peanut plant in the control area was 15.9-40.9 g, with an average value of 26.8 g, and the aboveground dry weight per peanut plant in the microbial agent treatment area was 28.6-41.8 g, with an average value of 34.1 g, and the dry weight per plant in the treatment area was increased by 27.2% compared with the control area.

The results show that microbial agent treatment has the effect of increasing the aboveground and underground biomass and dry matter accumulation of leguminous crops such as peanuts, thereby improving crop yield and quality.

The microbial agent developed by mixing Bacillus amyloliquefaciens, Brevibacillus laterosporu, Bacillus mucilaginosus Krassilnikov, and Enterobacter ludwigii according to the present disclosure can effectively increase the number of root nodules in leguminous crops such as peanuts, improve the nitrogenase activity and biological nitrogen fixation effect of root nodules, promote crop growth, increase the chlorophyll content in leaves, and aboveground and underground fresh weight, promote dry matter accumulation, and improve crop yield and quality, and has obvious economic and ecological benefits, has broad application prospects in leguminous crops such as peanuts, soybeans, alfalfa, and Chinese milk vetch, and is of great significance for reduction of fertilizer application in farmlands, peak carbon dioxide emissions, carbon neutrality and high-quality development in the agricultural industry.

What is claimed is:

1. A microbial agent for promoting root nodule number increase and root nodule nitrogenase activity increase in leguminous crops, compounded from 4 microbes comprising Bacillus amyloliquefaciens, Brevibacillus laterosporu, Bacillus mucilaginosus Krassilnikov, and Enterobacter ludwigii by separate fermentative cultivation, concentration and mixing;
the Bacillus amyloliquefaciens is a Bacillus amyloliquefaciens BA-HZ54 strain with an accession number of CCTCC NO: M 20211295; the Brevibacillus laterosporu is a Brevibacillus laterosporu BL-TS08 strain with an accession number of CCTCC NO: M 20211296; and the Bacillus mucilaginosus Krassilnikov is a Bacillus mucilaginosus Krassilnikov BM-TS05 strain with an accession number of CCTCC NO: M 20211297.

2. The microbial agent according to claim 1, wherein the Enterobacter ludwigii is an Enterobacter ludwigii BG10-1 strain with an accession number of CCTCC NO: M 2016014.

3. The microbial agent according to claim 1, having a living bacteria count of the Bacillus amyloliquefaciens being 2×10⁹ cfu/g or more, a living bacteria count of the Brevibacillus laterosporu being 2×10⁹ cfu/g or more, a living bacteria count of the Bacillus mucilaginosus Krassilnikov being 1×10¹⁰ cfu/g or more, and a living bacteria count of the Enterobacter ludwigii being 1×10¹⁰ cfu/g or more.

4. The microbial agent according to claim 1, wherein the microbial agent is a highly concentrated live bacterial granule or powder or aqueous agent.

5. The microbial agent according to claim 4, wherein a carrier of the granule comprises humic acid, tapioca flour and a bentonite binder in a weight ratio of 8.5:10:0.5, and raw materials are uniformly mixed and granulated and oven-dried to obtain a particle carrier for standby application.

6. The microbial agent according to claim 5, wherein the microbial agent is a granule produced by compounding concentrated bacterial solutions of strains with the carrier of the granule.

7. Use of the microbial agent according to claim 1 in promoting an increase of the number of root nodules and a nitrogenase activity in root nodules in leguminous crops.

8. The use according to claim 7, wherein a specific application method comprises applying the microbial agent at a usage amount of 2-4 kg/mu at a sowing stage or a growth period of the leguminous crops.

9. The use according to claim 7, wherein the leguminous crops comprise, but are not limited to, peanuts, soybeans, alfalfa, and Chinese milk vetch.

10. The use according to claim 7, further comprising inducing supernodulation of the leguminous crops.
